(19) United States
(12) Patent Application Publication
(10) Pub. No.: US 2008/0292573 A1
(43) Pub. Date: Nov. 27, 2008

(54) Method for treating hair with a reactive vinyl silicone capable of reacting via hydrosilylation

(76) Inventor: Franck Giroud, Chamoux sur Gelon (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/000,886

(22) Filed: Dec. 18, 2007

Related U.S. Application Data
(60) Provisional application No. 60/898,745, filed on Feb. 1, 2007.

(30) Foreign Application Priority Data
Dec. 20, 2006 (FR) 0655754

(51) Int. Cl. A61K 8/89; A61Q 5/00

(52) U.S. Cl. 424/70.12

(57) ABSTRACT

The subject of the present invention is a method for treating the hair which consists in applying to the hair at least one compound X and at least one compound Y, at least one of the compounds X and Y being a silicone compound, said compounds X and Y being able to react together via a hydrosilylation reaction.

The subject of the invention is also the use of the compounds X and Y for treating the hair, in particular for obtaining a coating of the hair which is resistant to shampooing operations.

The invention makes it possible to obtain hair that is particularly shiny and soft in a manner that is retained after several shampooing operations.

The subject of the present invention is a method for treating hair starting from a vinyl silicone capable of reacting via hydrosilylation on the hair.

The hair is generally damaged and embrittled by the action of external atmospheric agents, such as light and bad weather, and by mechanical or chemical treatments, such as brushing, combing, bleaching, perming and/or dyeing. The result of this is that the hair is often difficult to manage, in particular it is difficult to disentangle or to style, and heads of hair, even thick heads of hair, retain with difficulty a style which is attractive in appearance due to the fact that the hair lacks vigour, volume and liveliness.

Thus, in order to overcome this, it is now common to use styling products which make it possible to condition the hair by providing it, in particular, with body, bulk, sheen or volume. These styling products are generally cosmetic hair compositions comprising one or more polymers which have an affinity for the hair and which usually have the role of forming a film at the hair's surface for the purpose of modifying its surface properties, especially for conditioning it. To obtain such an effect, it is known, in particular, to use polysiloxanes, especially those described in documents FR 2 799 955, FR 2 799 956, FR 2 799 970 and FR 2 799 971.

One disadvantage linked to the use of these hair compositions lies in the fact that the cosmetic effects conferred by such compositions have a tendency to disappear, especially after the first shampooing operation.

In order to overcome this disadvantage, it is possible to envisage increasing the retention of the polymer deposit by directly carrying out a polymerization of certain monomers on the hair. Document U.S. Pat. No. 4,344,763 describes a hair setting composition that starts from a reactive aminoalkylalkoxysilane silicone and a titanate ester. It is also known to carry out coatings of the hair starting from a composition comprising an electrophilic monomer of cyanoacrylate type, especially in Patent Application FR 2 833 489. Such a composition makes it possible to obtain perfectly coated and non-greasy hair. However, the coating obtained does not give complete satisfaction with regard to external agents such as washing and perspiration. Furthermore, the coating obtained is sensitive to fatty substances such as sebum.

Documents WO 01/96450, GB 2 407 496 and EP 465 744 describe the use of particular reactive silicones for producing a film on the skin. Document WO 01/96450 and GB 2 407 496 describe a one-part formulation which comprises a polysiloxane having trialkoxyalkylsilyl end groups, a catalyst, a solvent and optionally an alkoxy-silane and fillers. These compositions make it possible to obtain, via condensation, a film on the skin. Document EP 465 744 describes the use of a polysiloxane with unsaturated aliphatic groups to produce medical devices for topical use.

The objective of the present invention is to develop a novel hair treatment method which makes it possible to obtain a long-lasting form retention of the hair while retaining good cosmetic properties.

Thus, the subject of the invention is a method for treating the hair which consists in applying to the hair at least one compound X and at least one compound Y, at least one of the compounds X and Y being a silicone compound, said compounds X and Y being able to react together via a hydrosilylation reaction.

The subject of the invention is also the use of the compounds X and Y described above for treating the hair, in particular for obtaining a coating which is resistant to shampooing operations.

The method of the invention makes it possible to obtain, in situ, a retained coating that is homogeneous, smooth and has an excellent adhesion to the hair. Furthermore, it has surprisingly been observed that the hair remained perfectly individualized, could be styled without any problems and that the styling properties provided to the hair were retained after shampooing operations.

### Compounds X and Y

The expression “silicone compound” is understood to mean a compound comprising at least two organosiloxane units. According to one particular embodiment, the compounds X and the compounds Y are silicone-based. The compounds X and Y may be aminated or non-aminated. They may comprise polar groups which may be chosen from the following groups: —COOH, —COO—, —COO—, —OH, —NH₂, —NH—, —NR—, —SO₃H, —SO₃, —OCH₂CH₂—, —O—CH₂CH₂CH₂—, —O—CH₂CH(CH₃)—, —NR₃⁺—, —SH, —NO₂, I, Cl, Br, —CN, —PO₄³⁻, —CONH—, —CONR—, —CONH₂, —CSNH—, —SO₂—, —SO—, —SO₂NH—, —NHCO—, —NHSO₂—, —NHCOO—, —OCONH—, —NHCSO— and —OCSNH—, R representing an alkyl group.

According to another embodiment, at least one of the compounds X and Y is a polymer whose main chain is predominantly formed from organosiloxane units.

Among the silicone compounds cited hereinbelow, some may have both film-forming and adhesive properties, depending, for example, on their proportion of silicone or depending on whether they are used in a mixture with a particular additive. It is consequently possible to adjust the film-forming properties or the adhesive properties of such compounds depending on the envisaged use, this is, in particular, the case for reactive silicone elastomers known as “room temperature vulcanization” silicones.

The compounds X and Y may react together at a temperature that varies between ambient temperature and 180° C. Advantageously, the compounds X and Y are able to react together at ambient temperature (20±5° C.) and atmospheric pressure, advantageously in the presence of a catalyst, via a hydrosilylation reaction or a condensation reaction, or a crosslinking reaction in the presence of a peroxide.

### 1—Compounds X and Y Capable of Reacting Via Hydrosilylation

According to one embodiment, the compounds X and Y are capable of reacting together via hydrosilylation, this reaction possibly being represented, in a simplified manner, as follows:

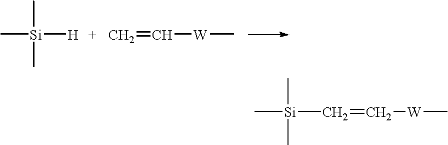

with W representing a carbon-based and/or silicone-based chain containing one or more unsaturated aliphatic groups.

In this case, the compound X may be chosen from silicone compounds comprising at least two unsaturated aliphatic groups. By way of example, the compound X may comprise a main silicone chain whose unsaturated aliphatic groups are pendant to the main chain (side group) or located at the ends of the main chain of the compound (end group). These particular compounds will be referred to, in the remainder of the description, as polyorganosiloxanes with unsaturated aliphatic groups. According to one embodiment, the compound X is chosen from polyorganosiloxanes comprising at least two unsaturated aliphatic groups, for example two or three vinyl or allyl groups, each bonded to a silicon atom. According to one advantageous embodiment, the compound X is chosen from polyorganosiloxanes comprising siloxane units of formula:

$${R}_{m} {R}^{′} SiO \frac{( 3 - m )}{2} \qquad ( I )$$

in which:
R represents a linear or cyclic, monovalent hydrocarbon-based group comprising from 1 to 30 carbon atoms, preferably from 1 to 20, and better still from 1 to 10 carbon atoms, such as, for example, a short-chain alkyl radical, for example, comprising from 1 to 10 carbon atoms, in particular a methyl radical or else a phenyl group, but preferably a methyl radical;
m is equal to 1 or 2; and
R′ represents:
   an unsaturated aliphatic hydrocarbon-based group comprising from 2 to 10, preferably from 2 to 5 carbon atoms such as, for example, a vinyl group or a —R″—CH═CHR′″ group in which R″ is a divalent aliphatic hydrocarbon-based chain comprising from 1 to 8 carbon atoms, bonded to the silicon atom, and R′″ is a hydrogen atom or an alkyl radical comprising from 1 to 4 carbon atoms, preferably a hydrogen atom; mention may be made, as an R′ group, of vinyl or allyl groups and mixtures thereof; or
   an unsaturated cyclic hydrocarbon-based group comprising from 5 to 8 carbon atoms such as, for example, a cyclohexenyl group.

Preferably, R′ is an unsaturated aliphatic hydrocarbon-based group, preferably a vinyl group.

According to one particular embodiment, the polyorgano-siloxane also comprises units of formula:

$${R}_{n} SiO \frac{( 4 - n )}{2} \qquad ( II )$$

in which R is a group such as defined above, and n is equal to 1, 2 or 3.

According to one variant, the compound X may be a silicone resin comprising at least two ethylenically unsaturated groups, said resin being capable of reacting with the compound B via hydrosilylation.

Mention may be made, for example, of the resins of MQ or MT type, that themselves bear unsaturated —CH═CH₂ reactive ends.

These resins are crosslinked organosiloxane polymers.

The nomenclature of silicone resins is known under the name “MDTQ”, the resin being described as a function of the various siloxane monomer units that it comprises, each of the letters “MDTQ” characterizing one type of unit.

The letter M represents the monofunctional unit of formula (CH₃)₃SiO_{1/2}, the silicon atom being bonded to a single oxygen atom in the polymer comprising this unit.

The letter D represents a difunctional unit (CH₃)₂SiO_{2/2} in which the silicon atom is bonded to two oxygen atoms.

The letter T represents a trifunctional unit of formula (CH₃) SiO_{3/2}

In the units M, D and T defined previously, at least one of the methyl groups may be substituted by an R group different from the methyl group such as a hydrocarbon-based (especially alkyl) radical having from 2 to 10 carbon atoms or a phenyl group or else a hydroxyl group.

Finally, the letter Q represents a tetrafunctional unit SiO_{4/2} in which the silicon atom is bonded to four hydrogen atoms themselves bonded to the rest of the polymer. As examples of such resins, mention may be made of MT silicone resins such as polyphenylvinylsilsesquioxanes such as those sold under the reference SST-3PV1 by Gelest.

Preferably, the compounds X comprise from 0.01 to 1% by weight of unsaturated aliphatic groups.

Advantageously, the compound X is chosen from polyorganopolysiloxanes, especially those comprising the siloxane units (I) and optionally (II) described previously.

The compound Y preferably comprises at least two free Si—H groups (hydridosilane groups).

The compound Y may advantageously be chosen from organosiloxanes comprising at least one alkylhydrogensiloxane unit of following formula:

$${R}_{p} HSiO \frac{( 3 - p )}{2} \qquad ( III )$$

in which:

R represents a linear or cyclic monovalent hydrocarbon-based group comprising from 1 to 30 carbon atoms, such as, for example, an alkyl radical having from 1 to 30 carbon atoms, preferably from 1 to 20 and better still from 1 to 10 carbon atoms, in particular a methyl radical, or else a phenyl group, and p is equal to 1 or 2. Preferably R is a hydrocarbon-based group, preferably methyl.

These compounds Y that are organosiloxanes with alkyl-hydrogensiloxane units may additionally comprise units of formula:

$${R}_{n} SiO \frac{( 4 - n )}{2} \qquad ( II )$$

such as defined above.

The compound Y may be a silicone resin comprising at least one unit chosen from the M, D, T and Q units such as defined above and comprising at least one Si—H group such as the polymethylhydrogensilsesquioxanes sold under the reference SST-3 MH1.1 by Gelest.

Preferably, these organosiloxane compounds Y comprise from 0.5 to 2.5% by weight of Si—H groups.

Advantageously, the R radicals represent a methyl group in the formulae (I), (II) and (III) above.

Preferably, these organosiloxanes Y comprise end groups of formula (CH₃)₃SiO_{1/2}.

Advantageously, the organosiloxanes Y comprise at least two alkylhydrogensiloxane units of formula (H₃C)(H)SiO and optionally comprise (H₃C)₂SiO units.

Such compounds Y that are organosiloxanes with hydrido-silane groups are described, for example, in document EP 0 465 744.

According to one variant, the compound X is chosen from organic oligomers or polymers (the term “organic” is understood to mean compounds whose main chain is not silicone-based, preferably compounds that do not comprise silicon atoms) or from organic/silicone hybrid polymers or oligomers, said oligomers or polymers bearing at least 2 reactive unsaturated aliphatic groups, the compound Y being chosen from the hydrogensiloxanes mentioned above.

The compound X, of organic nature, may then be chosen from vinyl or (meth)acrylic polymers or oligomers, polyesters, polyurethanes and/or polyureas, polyethers, perfluoropolyethers, polyolefins such as polybutene, polyisobutylene, organic hyperbranched dendrimers or polymers, or mixtures thereof.

In particular, the organic polymer or the organic part of the hybrid polymer may be chosen from the following polymers:

a) Polyesters with ethylenically unsaturated group(s):

These are a group of polymers of polyester type that have at least 2 ethylenic double bonds, distributed randomly in the main chain of the polymer. These unsaturated polyesters are obtained by polycondensation of a mixture:
of linear or branched aliphatic or cycloaliphatic carboxylic diacids comprising, in particular, from 3 to 50 carbon atoms, preferably from 3 to 20 and better still from 3 to 10 carbon atoms, such as adipic acid or sebacic acid, aromatic carboxylic diacids having, in particular, from 8 to 50 carbon atoms, preferably from 8 to 20 and better still from 8 to 14 carbon atoms, such as phthalic acids, especially terephthalic acid, and/or carboxylic diacids derived from fatty acid dimers with ethylenically unsaturated groups such as the dimers of oleic acid or linoleic acid described in Application EP-A-959 066 (paragraph [0021]) sold under the names Pripol® by Unichema or Empol® by Henkel, all these diacids having to be free of polymerizable ethylenic double bonds;
of linear or branched aliphatic or cycloaliphatic diols comprising, in particular, from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, aromatic diols having from 6 to 50 carbon atoms, preferably from 6 to 20 and better still from 6 to 15 carbon atoms, such as bisphenol A and bisphenol B, and/or diol dimers derived from the reduction of the fatty acid dimers such as defined previously; and
of one or more carboxylic diacids or their anhydrides comprising at least one polymerizable ethylenic double bond and having from 3 to 50 carbon atoms, preferably from 3 to 20 and better still from 3 to 10 carbon atoms, such as maleic acid, fumaric acid or itaconic acid.
   b) Polyesters with (meth)acrylate side and/or end groups:

These are a group of polymers of polyester type obtained by polycondensation of a mixture:
of linear or branched aliphatic or cycloaliphatic carboxylic diacids comprising, in particular, from 3 to 50 carbon atoms, preferably from 3 to 20 and better still from 3 to 10 carbon atoms, such as adipic acid or sebacic acid, aromatic carboxyllc diacids having, in particular, from 8 to 50 carbon atoms, preferably from 8 to 20 and better still from 8 to 14 carbon atoms, such as phthalic acids, especially terephthalic acid, and/or carboxylic diacids derived from fatty acid dimers with ethylenically unsaturated groups such as the dimers of oleic acid or linoleic acid described in Application EP-A-959 066 (paragraph [0021]) sold under the names Pripol® by Unichema or Empol® by Henkel, all these diacids having to be free of polymerizable ethylenic double bonds;
of linear or branched aliphatic or cycloaliphatic diols comprising, in particular, from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, aromatic diols having from 6 to 50 carbon atoms, preferably from 6 to 20 and better still from 6 to 15 carbon atoms, such as bisphenol A and bisphenol B; and of at least one ester of (meth)acrylic acid and a diol or polyol having from 2 to 20 carbon atoms, preferably from 2 to 6 carbon atoms, such as 2-hydroxyethyl (meth)acrylate, 2-hydroxypropyl (meth)acrylate and glycerol methacrylate.

These polyesters differ from those described above under point a) by the fact that the ethylenic double bonds are not located in the main chain but on the side groups or at the end of the chains. These ethylenic double bonds are those of the (meth)acrylate groups present in the polymer.

Such polyesters are sold, for example, by UCB under the names EBECRYL® (EBECRYL® 450: molar mass 1600, on average 6 acrylate functional groups per molecule, EBECRYL® 652: molar mass 1500, on average 6 acrylate functional groups per molecule, EBECRYL® 800: molar mass 780, on average 4 acrylate functional groups per molecule, EBECRYL® 810: molar mass 1000, on average 4 acrylate functional groups per molecule and EBECRYL® 50 000: molar mass 1500, on average 6 acrylate functional groups per molecule).

c) Polyurethanes and/or polyureas having (meth)acrylate groups, obtained by polycondensation:
of aliphatic, cycloaliphatic and/or aromatic diisocyanates, triisocyanates and/or polyisocyanates having, in particular, from 4 to 50, preferably from 4 to 30 carbon atoms, such as hexamethylene diisocyanate, isophorone diisocyanate, toluene diisocyanate, diphenylmethane diisocyanate or isocyanurates of formula:

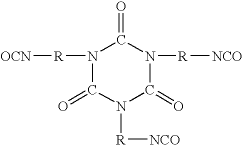

resulting from the trimerization of 3 OCN—R—CNO diisocyanate molecules, where R is a linear, branched or cyclic hydrocarbon-based radical comprising from 2 to 30 carbon atoms;
of polyols, especially diols, free of polymerizable ethylenically unsaturated groups, such as 1,4-butanediol, ethylene glycol or trimethylol-propane, and/or of aliphatic, cycloaliphatic and/or aromatic polyamines, especially diamines, having, in particular, from 3 to 50 carbon atoms, such as ethylenediamine or hexamethylenediamine; and
of at least one ester of (meth)acrylic acid and a diol or polyol having from 2 to 20 carbon atoms, preferably from 2 to 6 carbon atoms, such as 2-hydroxyethyl (meth)acrylate, 2-hydroxypropyl (meth)acrylate and glycerol methacrylate.

Such polyurethanes/polyureas having acrylate groups are sold, for example, under the name SR 368 (tris(2-hydroxyethyl)isocyanurate triacrylate) or CRAYNOR® 435 by Cray Valley, or under the name EBECRYL® by UCB (EBECRYL® 210: molar mass 1500, 2 acrylate functional groups per molecule, EBECRYL® 230: molar mass 5000, 2 acrylate functional groups per molecule, EBECRYL® 270: molar mass 1500, 2 acrylate functional groups per molecule, EBECRYL® 8402: molar mass 1000, 2 acrylate functional groups per molecule, EBECRYL® 8804: molar mass 1300, 2 acrylate functional groups per molecule, EBECRYL® 220: molar mass 1000, 6 acrylate functional groups per molecule, EBECRYL® 2220: molar mass 1200, 6 acrylate functional groups per molecule, EBECRYL® 1290: molar mass 1000, 6 acrylate functional groups per molecule, EBECRYL® 800: molar mass 800, 6 acrylate functional groups per molecule).

Mention may also be made of water-soluble aliphatic diacrylate polyurethanes sold under the names EBECRYL® 2000, EBECRYL® 2001 and EBECRYL® 2002, and diacrylate polyurethanes as an aqueous dispersion sold under the trade names IRR® 390, IRR® 400, IRR® 422, IRR® 424 by UCB.

d) Polyethers with (meth)acrylate groups obtained by esterification, via (meth)acrylic acid, of the hydroxyl end groups of C₁-C₄ alkylene glycol homopolymers or copolymers such as polyethylene glycol, polypropylene glycol, ethylene oxide/propylene oxide copolymers preferably having a weight-average molecular weight of less than 10 000, polyethoxylated or polypropoxylated trimethylolpropane.

Polyoxyethylene di(meth)acrylates of suitable molar mass are sold, for example, under the names SR 259, SR 344, SR 610, SR 210, SR 603 and SR 252 by Cray Valley or under the name EBECRYL® 11 by UCB. Polyethoxylated trimethylolpropane triacrylates are sold, for example, under the names SR 454, SR 498, SR 502, SR 9035 and SR 415 by Cray Valley or under the name EBECRYL® 160 by UCB. Polypropoxylated trimethylol-propane triacrylates are sold, for example, under the names SR 492 and SR 501 by Cray Valley.

e) Epoxy acrylates obtained by reaction between:

at least one diepoxide chosen, for example, from:
(i) bisphenol A diglycidyl ether;
(ii) a diepoxy resin resulting from the reaction between bisphenol A diglycidyl ether and epichlorohydrin;
(iii) an epoxy ester resin with α,ω-diepoxy end groups resulting from the condensation of a carboxylic diacid having from 3 to 50 carbon atoms with a stoichiometric excess of (i) and/or (ii);
(iv) an epoxy ether resin with α,ω-diepoxy end groups resulting from the condensation of a diol having from 3 to 50 carbon atoms with a stoichiometric excess of (i) and/or (ii);
(v) natural or synthetic oils bearing at least 2 epoxide groups, such as epoxidized soybean oil, epoxidized linseed oil and epoxidized vernonia oil;
(vi) a phenol/formaldehyde polycondensate (Novolac® resin), of which the end groups and/or the side groups have been epoxidized;
   and
   one or more carboxylic acids or polycarboxylic acids comprising at least one ethylenic double bond in the α,β position of the carboxyl group such as (meth)acrylic acid or crotonic acid or esters of (meth)acrylic acid and a diol or polyol having from 2 to 20 carbon atoms, preferably from 2 to 6 carbon atoms such as 2-hydroxyethyl (meth)acrylate.

Such polymers are sold, for example, under the names SR 349, SR 601, CD 541, SR 602, SR 9036, SR 348, CD 540, SR 480, CD 9038 by Cray Valley, under the names EBECRYL® 600 and EBECRYL® 609, EBECRYL® 150, EBECRYL® 860, EBECRYL® 3702 by UCB and under the names PHOTOMER® 3005 and PHOTOMER® 3082 by Henkel. f) Poly(C₁₋₅₀ alkyl (meth)acrylate)s, said alkyl being linear, branched or cyclic, comprising at least two functional groups with an ethylenic double bond borne by the hydrocarbon-based side and/or end chains.

Such copolymers are sold, for example, under the names IRR® 375, OTA® 480 and EBECRYL® 2047 by UCB.

g) Polyolefins such as polybutene and polyisobutylene.
h) Perfluoropolyethers with acrylate groups obtained by esterification, for example via (meth)acrylic acid, of perfluoropolyethers bearing hydroxyl side and/or end groups.

Such α,ω-diol perfluoropolyethers are especially described in EP-A-1 057 849 and are sold by Ausimont under the name FOMBLIN® Z DIOL.

i) Hyperbranched dendrimers and polymers bearing (meth)acrylate or (meth)acrylamide end groups obtained respectively by esterification or amidification of hyperbranched dendrimers and polymers having hydroxyl or amino end functional groups, via (meth)acrylic acid.

Dendrimers (from the Greek dendron=tree) are “arborescent”, that is to say highly branched, polymer molecules invented by D. A. Tomalia and his team at the beginning of the 1990s (Donald A. Tomalia et al., Angewandte Chemie, Int. Engl. Ed., Vol. 29, No. 2, pages 138-175). These are structures built around a central, generally polyvalent, unit. Linked around this central unit are, according to a perfectly determined structure, branched chain elongation units that thus give rise to symmetric monodisperse macromolecules having a well defined chemical and stereochemical structure. Polyamidoamine type dendrimers are sold, for example, under the name STARBURST® by Dendritech.

Hyperbranched polymers are polycondensates, generally of polyester, polyamide or polyethyleneamine type, obtained from multifunctional monomers, which have an arborescent structure similar to that of the dendrimers but much less regular than the latter (see, for example, WO-A-93/17060 and WO 96/12754).

The company Perstorp sells hyperbranched polyesters under the name BOLTORN®. Hyperbranched polyethylene-amines are found under the name COMBURST® from Dendritech. Hyperbranched polyesteramides with hydroxyl end groups are sold by DSM under the name HYBRANE®.

These hyperbranched dendrimers and polymers esterified or amidified by acrylic acid and/or methacrylic acid are differentiated from the polymers described under points a) to h) above by the very large number of ethylenic double bonds present. This high functionality, usually greater than 5, makes them particularly useful by allowing them to act as a “crosslinking node”, that is to say site of multiple crosslinking.

It is therefore possible to use these dendritic and hyperbranched polymers in combination with one or more of the polymers and/or oligomers a) to h) above.

### 1a Additional Reactive Compounds

According to one embodiment, the composition according to the invention may moreover comprise an additional reactive compound such as:
organic or inorganic particles comprising, at their surface, at least 2 unsaturated aliphatic groups, mention may be made, for example, of silicas surface-treated, for example, by silicone compounds with vinyl groups such as, for example, cyclotetramethyltetravinylsiloxane-treated silica; and
silazane compounds such as hexamethyldisilazane.

### 1b Catalyst

The hydrosilylation reaction is advantageously carried out in the presence of a catalyst which may be present in the composition according to the invention, the catalyst preferably being based on platinum or on tin.

Mention may be made, for example, of catalysts based on platinum deposited on a support of silica gel or of charcoal powder, platinum chloride, platinum salts and chloroplatinic acids.

Chloroplatinic acids in hexahydrate or anhydrous form, which are easily dispersible in the organosilicone media, are preferably used.

Mention may also be made of platinum complexes such as those based on chloroplatinic acid hexahydrate and divinyltetramethyldisiloxane.

The catalyst may be present in the composition according to the present invention in an amount ranging from 0.0001% to 20% by weight relative to the total weight of the composition.

It is also possible to introduce polymerization inhibitors or retardants, and more particularly catalyst inhibitors, into the composition of the invention, this being in order to increase the stability of the composition over time or to retard the polymerization. In a non-limiting manner, mention may be made of cyclic polymethylvinylsiloxanes, and in particular tetravinyltetramethylcyclotetrasiloxane, acetylenic alcohols, which are preferably volatile, such as methylisobutynol.

The presence of ionic salts, such as sodium acetate, in the composition may have an influence on the polymerization rate of the compounds.

Advantageously, the compounds X and Y are chosen from silicone compounds capable of reacting via hydrosilylation; in particular the compound X is chosen from polyorganosiloxanes comprising units of formula (I) described above and the compound Y is chosen from organosiloxanes comprising alkylhydrogensiloxane units of formula (III) described above.

According to one particular embodiment, the compound X is a polydimethylsiloxane with vinyl end groups and the compound Y is methylhydrogensiloxane.

By way of example of a combination of compounds X and Y that reacts via hydrosilylation, mention may be made of the following references proposed by Dow Corning: DC 7-9800 Soft Skin Adhesive Parts A & B, and also the following mixtures A and B prepared by Dow Corning:

### Mixture A:

| | | Content | |
|---|---|---|---|
| Ingredient (INCI name) | CAS No. | (%) | Function |
| Dimethyl Siloxane, | 68083-19-2 | 55-95 | Polymer |
| Dimethylvinylsiloxy-terminated | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

### Mixture B:

| | | Content | |
|---|---|---|---|
| Ingredient (INCI name) | CAS No. | (%) | Function |
| Dimethyl Siloxane, | 68083-19-2 | 55-95 | Polymer |
| Dimethylvinylsiloxy-terminated | | | |
| Silica Silylate | 68909-20-6 | 10-40 | Filler |
| Dimethyl, Methylhydrogen | 68037-59-2 | 1-10 | Polymer |
| Siloxane, trimethylsiloxy- | | | |
| terminated | | | |

According to one embodiment, the hydrosilylation reaction between the compounds X and Y is accelerated by a supply of heat, for example by raising the temperature of the system to between 25° C. and 180° C. The system will react, in particular, on keratinous fibres.

Generally, the compounds X and Y react together, the molar percentage of X relative to the total of compounds X and Y, that is to say the X/(X+Y)×100 ratio, may vary from 5% to 95%, preferably from 10% to 90% and better still from 20% to 80%.

Similarly, the molar percentage of Y relative to the total of compounds X and Y, that is to say the Y/(X+Y)×100 ratio, may vary from 5% to 95%, preferably from 10% to 90% and better still from 20% to 80%.

The compound X may have a weight-average molecular weight (M_{w}) ranging from 150 to 1 000 000, preferably from 200 to 800 000, more preferably from 200 to 250 000.

The compound Y may have a weight-average molecular weight (M_{w}) ranging from 200 to 1 000 000, preferably from 300 to 800 000, more preferably from 500 to 250 000.

The compound X may represent from 0.5% to 95%, preferably from 1% to 90% and better still from 5% to 80%, by weight relative to the total weight of the composition.

The compound Y may represent from 0.05% to 95%, preferably from 0.1% to 90% and better still from 0.2% to 80%, by weight relative to the total weight of the composition.

The ratio between the compounds X and Y, when X and Y are different, may be varied so as to adjust the reaction rate and therefore the film-formation rate or else so as to adapt the properties of the film formed (for example its adhesive properties) according to the desired application.

In particular the compounds X and Y may be present in an X/Y molar ratio ranging from 0.05 to 20 and better still from 0.1 to 10.

According to one variant, the method of the invention comprises two steps, a first step which consists in applying, to the hair, at least one composition comprising the compound X and a second step which consists in applying at least one composition comprising the compound Y, the steps being able to be reversed.

When the catalyst is present, it may be either in the composition containing X or in the composition comprising Y, or in both compositions, or in a third composition, the three compositions being able to be applied in any order.

According to another variant, the method of the invention comprises a first step that consists in applying a composition comprising X and Y, and a second composition comprising the catalyst.

According to the method of the invention, it is possible to apply to the hair, several times in alternation, a composition comprising X and a composition comprising Y.

The method of the present invention may comprise one or more additional steps such as the application of a film-forming polymer, a fixing polymer, a conditioning agent, a washing step or a drying step.

The composition of the invention may contain water or one or more organic solvents, or a mixture of water and one or more organic solvents.

The expression “organic solvent” is understood to mean an organic substance that is liquid at a temperature of 25° C. at atmospheric pressure (760 mm Hg) capable of dissolving another substance without chemically modifying it.

The organic solvent or solvents that can be used in the present invention are different from the compounds X and Y defined previously.

The organic solvent is, for example, chosen from aromatic alcohols such as benzyl alcohol; liquid, especially C₁₀-C₃₀, fatty alcohols, modified or unmodified polyols such as glycerol, glycol, propylene glycol, dipropylene glycol, butylene glycol, butyl diglycol; volatile silicones such as short-chain linear silicones like hexamethyldisiloxane and octamethyltrisiloxane, cyclic silicones, such as octamethylcyclotetrasiloxane, decamethylcyclopentasiloxane, dodecamethylcyclohexasiloxane, polydimethylsiloxanes optionally modified by alkyl and/or amine and/or imine and/or fluoroalkyl and/or carboxylic and/or betaine and/or quaternary ammonium functional groups, liquid modified polydimethylsiloxanes, mineral, organic or plant oils, alkanes and more particularly C₅ to C₁₀ alkanes; liquid fatty acids, liquid fatty esters and more particularly liquid fatty alcohol benzoates or salicylates.

The organic solvent is preferably chosen from organic oils; silicones such as volatile silicones, aminated or non-aminated silicone oils or gums, and mixtures thereof; mineral oils; plant oils such as olive oil, castor oil, rapeseed oil, coconut oil, wheat germ oil, sweet almond oil, avocado oil, macadamia oil, apricot oil, safflower oil, candlenut oil, camelina oil, tamanu oil, lemon oil or else organic compounds such as C₅-C₁₀ alkanes, acetone, methyl ethyl ketone, esters of liquid C₁-C₂₀ acids and C₁-C₈ alcohols such as methyl acetate, butyl acetate, ethyl acetate and isopropyl myristate, dimethoxyethane, diethoxyethane, liquid C₁₀-C₃₀ fatty alcohols such as oleyl alcohol, esters of fatty alcohols or of fatty acids such as C₁₀-C₃₀ fatty alcohol benzoates and mixtures thereof; isononyl isononanoate, isostearyl malate, pentaerythritol tetraisostearate, tridecyl trimelate; polybutene oil; the cyclopentasiloxane (14.7 wt %)/α,γ-dihydroxylated polydimethylsiloxane (85.3 wt %) mixture, or mixtures thereof.

According to one preferred embodiment, the organic solvent is composed of a silicone or a mixture of silicones such as liquid polydimethylsiloxanes and modified liquid polydimethylsiloxanes, the viscosity of the silicone and/or of the mixture of silicones at 25° C. is between 0.1 cST and 1 000 000 cST and more preferably between 1 cST and 30 000 cST.

Mention will preferably be made of the following oils and mixtures of oils:
the α,ω-dihydroxylated polydimethylsiloxane/cyclopentadimethylsiloxane (14.7/85.3) mixture sold by Dow Corning under the name DC 1501 Fluid;
the α,ω-dihydroxylated polydimethylsiloxane/polydimethylsiloxane mixture sold by Dow Corning under the name DC 1503 Fluid;
the dimethicone/cyclopentadimethylsiloxane mixture sold by Dow Corning under the name DC 1411 Fluid or that sold by Bayer under the name SF 1214;
the cyclopentadimethylsiloxane sold by Dow Corning under the name DC 245 Fluid; and the respective mixtures of these oils.

The organic solvent or solvents and water when it is present generally represent from 0.01 to 99%, preferably from 50 to 99% by weight relative to the total weight of the composition.

The composition of the invention may contain, besides the organic solvent or solvents, water in proportions varying from 1 to 99%, preferably from 1 to 50% relative to the total weight of the composition. However, according to one particular embodiment, the composition of the invention is anhydrous, that is to say containing less than 1% by weight of water relative to the total weight of the composition.

The composition of the invention may also be in the form of an emulsion and/or be encapsulated. When the composition is an emulsion, it is for example composed of a dispersed or continuous phase which may be water, C₁-C₄ aliphatic alcohols or mixtures thereof and an organic phase that is not soluble in water.

The composition according to the invention may also contain, besides the compounds X and Y, at least one agent commonly used in cosmetics chosen, for example, from reducing agents, fatty substances, plasticizers, softeners, anti-foaming agents, moisturizers, pigments, clays, mineral fillers, UV-screening agents, mineral colloids, peptizers, fragrances, preservatives, anionic, cationic, non-ionic or amphoteric surfactants, fixing or non-fixing polymers, proteins, vitamins, direct dyes other than the hydrophobic dyes of the invention, oxidation dyes, pearlescent agents, propellants, and mineral or organic thickeners such as benzylidene sorbitol and N-acyl amino acids, oxyethylenated or non-oxyethylenated waxes, paraffins, C₁₀-C₃₀ solid fatty acids such as stearic acid, lauric acid, fatty amides or solid fatty acids.

The compositions may be in various dosage forms such as a lotion, an aerosol mousse, a conditioner or a shampoo, a gel or a wax. The compositions may be contained in a pump dispenser or an aerosol spray. After application to the hair, the compositions of the invention may be rinsed out or left on.

When the composition is contained in an aerosol, it may contain a propellant. The propellant is composed of the compressed or liquefied gases normally used for preparing aerosol compositions. Use will preferably be made of air, carbon dioxide, compressed nitrogen or else a soluble gas such as dimethyl ether, halogenated (fluorinated in particular) hydrocarbons or non-halogenated hydrocarbons (butane, propane, isobutane) and mixtures thereof. Where necessary, it could be possible to use chambered aerosols containing one or more chambers.

The subject of the invention is also the use of the compounds X and Y as defined above, for treating the hair, in particular for giving the hair a coating which is retained.

The invention is illustrated in greater detail by the examples described below. Except where indicated otherwise, the amounts indicated are expressed as weight percentages.

### EXAMPLES

The following compositions were prepared from a mixture of polydimethylhydrogensiloxane and divinyl polydimethylsiloxane, with a platinum-based catalyst sold by Dow Corning under the name 7-9800 PART A and 7-9800 PART B.

Introduced into 90 g of ethanol were 5 g of Dow Corning 7-9800 PART A and 5 g of Dow Corning 7-9800 PART B. The two silicone compounds were then emulsified in the ethanol using a Turax type turbo mixer.

10 g of the preceding emulsion were applied to half of a head formed of mid-length natural Caucasian hair and 10 g of ethanol were applied to the other half of the head. The head of hair thus treated was left overnight at ambient temperature.

The whole of the head was then washed with a mild shampoo.

After 5 shampooing operations, it was noted that the part of the head of hair treated according to the method of the present invention was shinier, could be styled more easily and the hair was softer and had more body than the part of the head of hair treated only with ethanol.

In conclusion, the application of a reactive silicone of the invention provides beneficial cosmetic effects and this in a manner that is retained after shampooing operations.

1. Method for treating the hair which consists in applying to the hair at least one compound X and at least one compound Y, at least one of the compounds X and Y being a silicone compound, said compounds X and Y being able to react together via a hydrosilylation reaction.

2-29. (canceled)
